(19) United States
(12) Patent Application Publication
(10) Pub. No.: US 2024/0065872 A1
(43) Pub. Date: Feb. 29, 2024

(54) Back Brace Assembly

(71) Applicants: Kris Andrues, Vallejo, CA (US); Stephen Andrues, Vallejo, CA (US)

(72) Inventors: Kris Andrues, Vallejo, CA (US); Stephen Andrues, Vallejo, CA (US)

(21) Appl. No.: 17/895,217

(22) Filed: Aug. 25, 2022

(51) Int. Cl. A61F 5/02

(52) U.S. Cl. CPC A61F 5/026

(57) ABSTRACT

A back brace assembly for assisting a user with standing upright includes a body harness that can be worn around a user's upper torso. The body harness includes a back panel which extends along the user's back when the body harness is worn. A pair of thigh cuffs is each worn around a respective one of the user's thighs. Each of the thigh cuffs is attachable to the body harness thereby inhibiting the back panel from moving beyond a pre-determined distance from the thigh cuffs to assist the user with standing upright. A pair of heel straps is each of the heel straps is releasably matable to a respective one of the thigh cuffs. Each of the heel straps has a loop is integrated therein which can be positioned around a respective one of the user's heels thereby enhancing stability of the user for standing upright.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to brace devices and more particularly pertains to a new brace device for assisting a user with standing upright. The device includes a body harness which is wearable around a user's upper torso. The device includes a pair of thigh cuffs that is each wearable around the user's thighs. Each of the thigh cuffs is attachable to the body harness for assisting the user with standing upright. A pair of heel straps is provided which each engage a respective one of the user's heels. Additionally, each of the heel straps is attachable to a respective thigh cuff for further assisting the user with standing upright.

### (2) Description of Related Art Including Information Disclosed Under 37 CFR 1.97 and 1.98

The prior art relates to brace devices including a back support device that includes a variety of back support devices which each at least includes a body harness and a pair of thigh cuffs that are attached to the body harness for assisting a user with standing upright. The prior art discloses a body support device that includes a body harness and a rigid member that is attached to the body harness for assisting a user with bending over and lifting. The prior art discloses a back support brace that includes shoulder straps that wrap around a user's shoulders, a waist strap and a back strap which extends between the shoulder straps and the waist strap. The prior art discloses a support garment which includes a pair of shorts and a shirt for providing support of a user's back. The p

### BRIEF SUMMARY OF THE INVENTION

An embodiment of the disclosure meets the needs presented above by generally comprising a body harness that can be worn around a user's upper torso. The body harness includes a back panel which extends along the user's back when the body harness is worn. A pair of thigh cuffs is each worn around a respective one of the user's thighs. Each of the thigh cuffs is attachable to the body harness thereby inhibiting the back panel from moving beyond a pre-determined distance from the thigh cuffs to assist the user with standing upright. A pair of heel straps is each of the heel straps is releasably matable to a respective one of the thigh cuffs. Each of the heel straps has a loop is integrated therein which can be positioned around a respective one of the user's heels thereby enhancing stability of the user for standing upright.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a back brace assembly according to an embodiment of the disclosure.

FIG. 2 is a front view of an embodiment of the disclosure.

FIG. 3 is a back view of an embodiment of the disclosure.

FIG. 4 is a front perspective in-use view of an embodiment of the disclosure.

FIG. 5 is a back perspective in-use view of an embodiment of the disclosure.

### DETAILED DESCRIPTION OF THE INVENTION

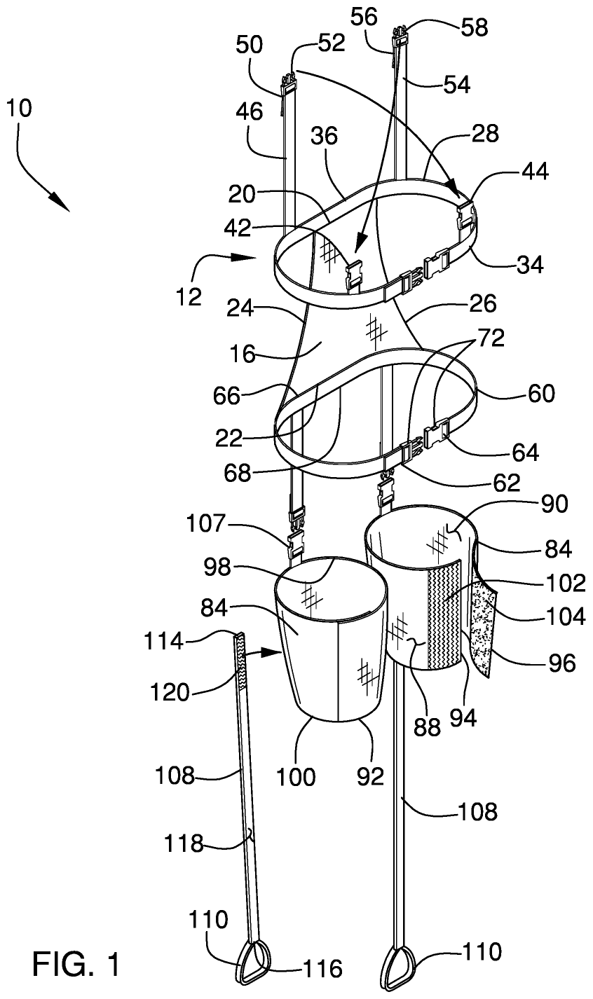

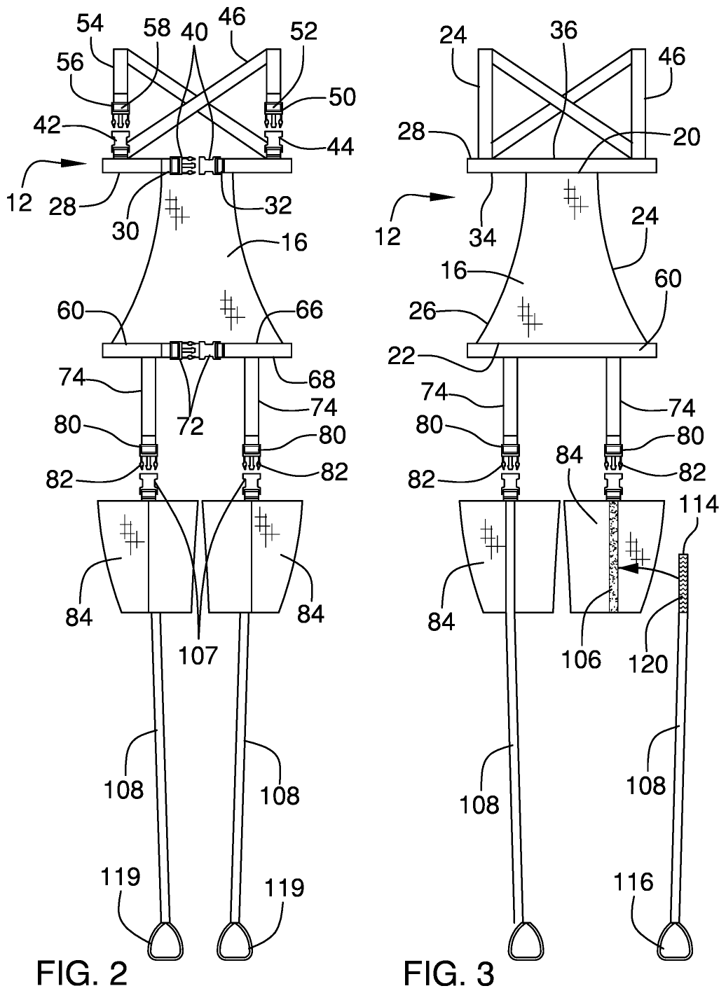

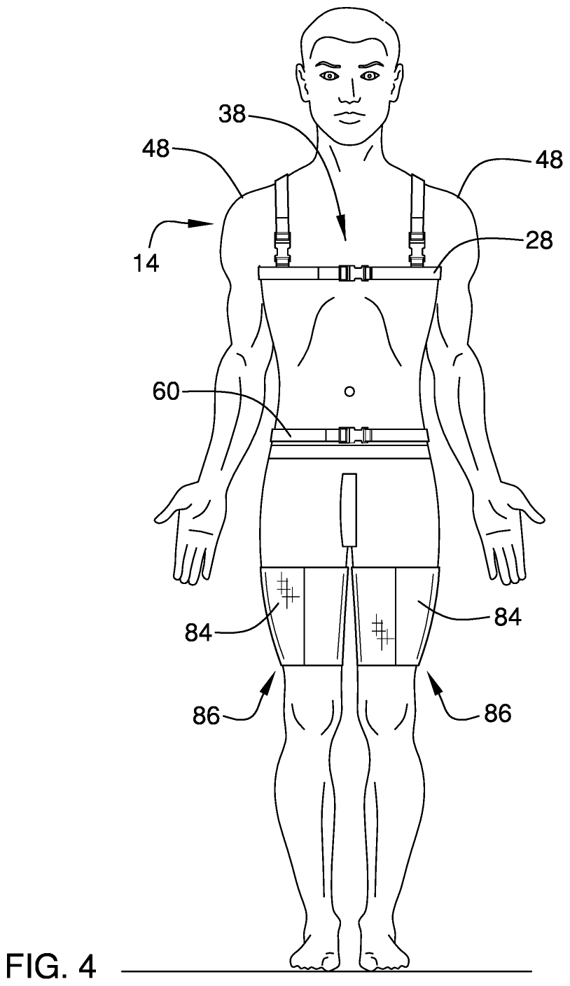

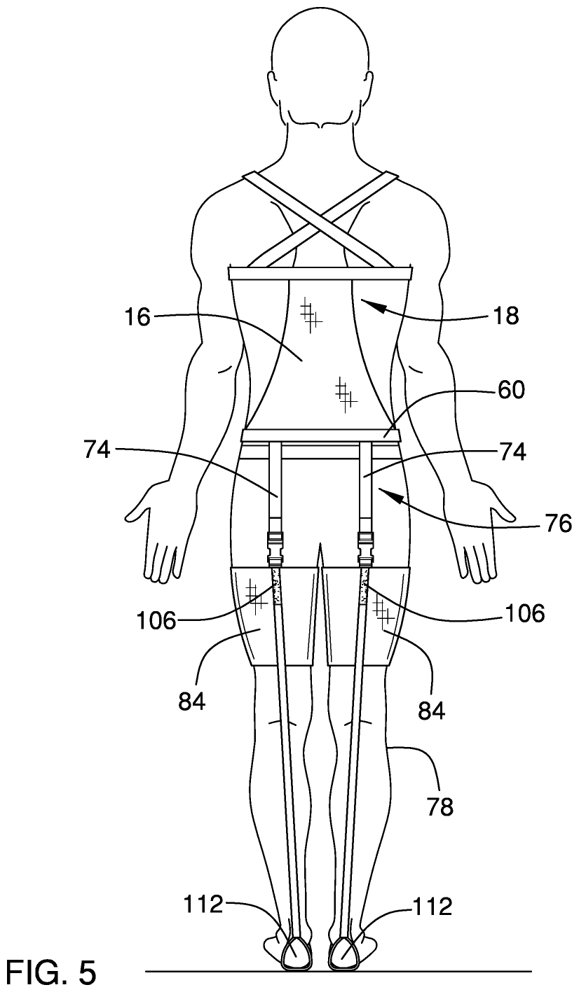

With reference now to the drawings, and in particular to FIGS. 1 through 5 thereof, a new brace device embodying the principles and concepts of an embodiment of the disclosure and generally designated by the reference numeral 10 will be described.

As best illustrated in FIGS. 1 through 5, the back brace assembly 10 generally comprises a body harness 12 that can be worn around a user's upper torso 14. The body harness 12 includes a back panel 16 that is integrated into the body harness 12. The back panel 16 extends along the user's back 18 when the body harness 12 is worn. The back panel 16 has a top edge 20, a bottom edge 22, a first lateral edge 24 and a second lateral edge 26. Each of the first lateral edge 24 and the second lateral edge 26 curves inwardly between the bottom edge 22 and the top edge 20 such that the bottom edge 22 has a length that is greater than a length of the top edge 20. The user may be suffering from a back injury or other back condition that reduces, or eliminates, the user's ability to stand upright.

The body harness 12 comprises a chest strap 28 that has a first end 30, a second end 32, a lower edge 34 and an upper edge 36. The chest strap 28 is elongated between the first end 30 and the second end 32 such that the chest strap 28 can be worn around the user's chest 38. Additionally, the lower edge 34 is attached to the top edge 20 of the back panel 16. A pair of chest couplers 40 is each coupled to a respective one of the first end and the second end 32 of the chest strap 28. Moreover, each of the chest couplers 40 is releasably matable to each other for retaining the chest strap 28 around the user's chest 38. Each of the chest couplers 40 may comprise a pair of complementary buckles or other type of releasable, mechanical coupler. Additionally, the chest strap 28 slidably engages each of the chest couplers 40 for adjusting a length of the chest strap 28 to accommodate the size of the user's chest 38.

A first coupler 42 is coupled to and extends upwardly from the upper edge 36 of the chest strap 28 and the first coupler 42 is spaced from the first end 30 of the chest strap 28. A second coupler 44 is coupled to and extends upwardly from the upper edge 36 of the chest strap 28. The second coupler 44 is spaced from the second end 32 of the chest strap 28. Each of the first coupler 42 and the second coupler 44 may comprise complementary buckles or other type of releasable, mechanical coupler.

A first shoulder strap 46 is coupled to and extends upwardly from the upper edge 36 of the chest strap 28 for wearing over a respective one of the user's shoulders 48. The first shoulder strap 46 has a distal end 50 with respect to the chest strap 28. A first shoulder coupler 52 is coupled to the distal end 50 of the first shoulder strap 46 and the first shoulder strap 46 is oriented to extend cross wise across the user's chest 38 having the first shoulder coupler 52 releasably engaging the second coupler 44. A second shoulder strap 54 is coupled to and extends upwardly from the upper edge 36 of the chest strap 28 for wearing over a respective one of the user's shoulders 48. The second shoulder strap 54 has a distal end 56 with respect to the chest strap 28. A second shoulder coupler 58 is coupled to the distal end 56 of the second shoulder strap 54. the second shoulder strap 54 is oriented to extend cross wise across the user's chest 38 having the second shoulder coupler 58 releasably engaging the first coupler 42. Each of the first shoulder coupler 52 and the second shoulder coupler 58 may comprise complementary buckles or other type of releasable, mechanical coupler.

A waist strap 60 has a primary end 62, a secondary end 64, an upper edge 66 and a lower edge 68. The waist strap 60 is elongated between the primary end 62 and the secondary end 64 such that the waist strap 60 can be worn around the user's waist 70. Furthermore, the upper edge 66 is coupled to the bottom edge of the back panel 16. A pair of waist couplers 72 is each coupled to a respective one of the primary end 62 and the secondary end 64 of the waist strap 60. Each of the waist couplers 72 is releasably matable to each other for retaining the waist strap 60 around the user's waist 70. Each of the waist couplers 72 may comprise complementary buckles or other type of releasable, mechanical coupler.

A pair of leg straps 74 is provided each of the leg straps 74 is coupled to and extends downwardly from the lower edge 68 of the waist strap 60. Each of the leg straps 74 extends downwardly along a back side 76 of a respective one of the user's legs 78. Each of the leg straps 74 has a distal end 80 with respect to the lower edge 68 of the waist strap 60. A pair of leg strap couplers 82 is provided and each of the leg strap couplers 82 is coupled to the distal end 80 of a respective one of the leg straps 74. Each of the leg strap couplers 82 may comprise complementary buckles or other type of releasable, mechanical coupler.

A pair of thigh cuffs 84 each is provided and each of the thigh cuffs 84 can be worn around a respective one of the user's thighs 86. Each of the thigh cuffs 84 is attachable to the body harness 12 thereby inhibiting the back panel 16 from moving beyond a pre-determined distance from the thigh cuffs 84. In this way the pair of thigh cuffs 84 can assist the user with standing upright. Each of the thigh cuffs 84 has a first surface 88, a second surface 90 and a perimeter edge 92 extending between the first surface 88 and the second surface 90. The perimeter edge 92 has a first lateral side 94, a second lateral side 96, a top side 98 and a bottom side 100, and each of the thigh cuffs 84 can be rolled onto themselves to form a cylinder. The top side 98 has a length is greater than a length of the bottom side 100 such that each of the cylinder formed by the thigh cuffs 84 tapers between the top side 98 and the bottom side 100 to the shape of the user's thighs 86.

A pair of first mating members 102 is provided and each of the first mating members 102 is coupled to the first surface 88 of a respective one of the thigh cuffs 84. Each of the first mating members 102 is aligned with the first lateral side 94 of the perimeter edge 92 of the respective thigh cuff 84. A pair of second mating members 104 is provided and each of the second mating members 104 is coupled to the second surface 90 of a respective one of the thigh cuffs 84. Each of the second mating members 104 is aligned with the second lateral side 96 of the perimeter edge 92 of the respective thigh cuff 84. Additionally, the first mating member 102 on a respective one of the thigh cuffs 84 is matable to the second mating member 104 on the respective thigh cuff 84. Each of the first mating member 102 and the second mating member 104 may comprise a hook and loop fastener or other type of multiple use, releasable fastener.

A pair of first heel strap mating members 106 is provided and each of the first heel strap mating members 106 is coupled to the first surface 88 of a respective one of the thigh cuffs 84. Each of the first heel strap mating members 106 extends between the top side 98 and the bottom side 100 of the perimeter edge 92 of the respective thigh cuff 84. A pair of thigh couplers 107 is provided and each of the thigh couplers 107 is coupled to the top side 98 of the perimeter edge 92 of a respective one of the thigh cuffs 84. Each of the thigh couplers 107 is releasably matable to a respective one of the leg strap couplers 82.

A pair of heel straps 108 is provided and each of the heel straps 108 is releasably matable to a respective one of the thigh cuffs 84. Each of the heel straps 108 has a loop 110 integrated therein such that the loop 110 on each of the heel straps 108 can be positioned around a respective one of the user's heels 112 thereby enhancing stability of the user for standing upright.

Each of the heel straps 108 has an upper end 114, a lower end 116 and a primary surface 118 extending between the upper end 114 and the lower end 116. Each of heel straps 108 has a stirrup 119 that is coupled to the lower end 116 having the stirrup 119 on each of the heel straps 108 defining the loop 110. In this way the stirrup 119 can be positioned around the respective user's heel 112. A pair of second heel strap mating members 120 is each coupled to the primary surface 118 of a respective one of the heel straps 108. Each of the second heel strap mating members 120 is matable to a respective one of the first heel strap mating members 106.

In use, the chest strap 28 is worn around the user's chest 38 and the waist strap 60 is worn around the user's waist 70. Each of the first shoulder strap 46 and the second shoulder strap 54 is worn over the user's shoulders 48 and each of the first shoulder strap 46 and the second shoulder strap 54 is crossed over the user's chest 38 and coupled to respective first shoulder coupler 52 and second shoulder coupler 58. Each of the thigh cuffs 84 is positioned around the respective user's thigh 86 and each of the thigh couplers 107 is attached to the respective leg strap 74. In this way the back panel 16 is retained a fixed distance from the thigh cuffs 84 to assist the user with standing upright. Each of the heel straps 108 is coupled to the respective thigh cuff 84 such that the stirrup 119 on each of the heel straps 108 is positioned a desired distance from the respective thigh cuff 84. In this way the tension imparted onto the thigh cuff 84 can either be increased or decreased, depending on the user's preference. Thus, the thigh cuffs 84, the heel straps 108 and the body harness 12 work in conjunction to assist the user with standing upright.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A back brace assembly for assisting a user suffering from a back injury to stand upright, said assembly comprising:
a body harness being configured to be worn around a user's upper torso, said body harness including a back panel being integrated into said body harness wherein said back panel is configured to extend along the user's back when said body harness is worn;
a pair of thigh cuffs each being configured to be worn around a respective one of the user's thighs, each of said thigh cuffs being attachable to said body harness thereby inhibiting said back panel from moving beyond a pre-determined distance from said thigh cuffs wherein said pair of thigh cuffs is configured to assist the user with standing upright;
a pair of thigh couplers, each of said thigh couplers being coupled to said top side of said perimeter edge of a respective one of said thigh cuffs, each of said thigh couplers being releasably matable to a respective one of said leg strap couplers; and
a pair of heel straps, each of said heel straps being releasably matable to a respective one of said thigh cuffs, each of said heel straps having a loop being integrated therein wherein said loop on each of said heel straps is configured to be positioned around a respective one of the user's heels thereby enhancing stability of the user for standing upright.

2. The assembly according to claim 1, wherein:
said back panel has a top edge, a bottom edge, a first lateral edge and a second lateral edge, each of said first lateral edge and said second lateral edge curving inwardly between said bottom edge and said top edge such that said bottom edge has a length being greater than a length of said top edge;
said body harness includes a chest strap having a first end, a second end, a lower edge and an upper edge, said chest strap being elongated between said first end and said second end wherein said chest strap is configured to be worn around the user's chest, said lower edge being attached to said top edge of said back panel; and
said body harness includes a pair of chest couplers, each of said chest couplers being coupled to a respective one of said first end and said second end of said chest strap, each of said chest couplers being releasably matable to each other for retaining said chest strap around the user's chest.

3. The assembly according to claim 2, wherein said body harness comprises:
a first coupler being coupled to and extending upwardly from said upper edge of said chest strap, said first coupler being spaced from said first end of said chest strap; and
a second coupler being coupled to and extending upwardly from said upper edge of said chest strap, said second coupler being spaced from said second end of said chest strap.

4. The assembly according to claim 3, wherein:
said body harness includes a first shoulder strap being coupled to and extending upwardly from said upper edge of said chest strap wherein said first shoulder strap is configured to be worn over a respective one of the user's shoulders, said first shoulder strap having a distal end with respect to said chest strap; and
said body harness includes a first shoulder coupler being coupled to said distal end of said first shoulder strap, said first shoulder strap being oriented to extend cross wise across the user's chest having said first shoulder coupler releasably engaging said second coupler.

5. The assembly according to claim 4, wherein:
said body harness includes a second shoulder strap being coupled to and extending upwardly from said upper edge of said chest strap wherein said second shoulder strap is configured to be worn over a respective one of the user's shoulders, said second shoulder strap having a distal end with respect to said chest strap; and
said body harness includes a second shoulder coupler being coupled to said distal end of said second shoulder strap, said second shoulder strap being oriented to extend cross wise across the user's chest having said second shoulder coupler releasably engaging said first coupler.

6. The assembly according to claim 2, wherein:
said body harness includes a waist strap having a primary end, a secondary end, an upper edge and a lower edge, said waist strap being elongated between said primary end and said secondary end wherein said waist strap is configured to be worn around the user's waist, said upper edge being coupled to said bottom edge of said back panel; and
said body harness includes a pair of waist couplers, each of said waist couplers being coupled to a respective one of said primary end and said secondary end of said waist strap, each of said waist couplers being releasably matable to each other for retaining said waist strap around the user's waist.

7. The assembly according to claim 6, wherein:
said body harness includes a pair of leg straps, each of said leg straps being coupled to and extending downwardly from said lower edge of said waist strap wherein each of said leg straps is configured to extend downwardly along a back side of a respective one of the user's legs, each of said leg straps having a distal end with respect to said lower edge of said waist strap; and
said body harness includes a pair of leg strap couplers, each of said leg strap couplers being coupled to said distal end of a respective one of said leg straps.

8. The assembly according to claim 7, wherein:
each of said thigh cuffs has a first surface, a second surface and a perimeter edge extending between said first surface and said second surface, said perimeter edge having a first lateral side, a second lateral side, a top side and a bottom side, each of said thigh cuffs being rolled onto themselves to form a cylinder, said top side having a length being greater than a length of said bottom side such that each of said cylinder formed by said thigh cuffs tapers between said top side and said bottom side wherein each of said thigh cuffs is configured to conform to the shape of the user's thighs; and
said assembly includes a pair of thigh couplers, each of said thigh couplers being coupled to said top side of said perimeter edge of a respective one of said thigh cuffs, each of said thigh couplers being releasably matable to a respective one of said leg strap couplers.

9. The assembly according to claim 8, further comprising:
a pair of first mating members, each of said first mating members being coupled to said first surface of a respective one of said thigh cuffs, each of said first mating members being aligned with said first lateral side of said perimeter edge of said respective thigh cuff; and
a pair of second mating members, each of said second mating members being coupled to said second surface of a respective one of said thigh cuffs, each of said second mating members being aligned with said second lateral side of said perimeter edge of said respective thigh cuff, said first mating member on a respective one of said thigh cuffs being matable to said second mating member on said respective thigh cuff.

10. The assembly according to claim 8, further comprising a pair of first heel strap mating members, each of said first heel strap mating members being coupled to said first surface of a respective one of said thigh cuffs, each of said first heel strap mating members extending between said top side and said bottom side of said perimeter edge of said respective thigh cuff.

11. The assembly according to claim 1, wherein each of said heel straps has an upper end, a lower end and a primary surface extending between said upper end and said lower end, each of heel straps having a stirrup being coupled to said lower end having said stirrup on each of said heel straps defining said loop wherein said stirrup is configured to be positioned around the respective user's heel.

12. The assembly according to claim 11, further comprising:
a pair of first heel strap mating members; and
a pair of second heel strap mating members, each of said second heel strap mating members being coupled to said primary surface of a respective one of said heel straps, each of said second heel strap mating members being matable to a respective one of said first heel strap mating members.

13. A back brace assembly for assisting a user suffering from a back injury to stand upright, said assembly comprising:
a body harness being configured to be worn around a user's upper torso, said body harness including a back panel being integrated into said body harness wherein said back panel is configured to extend along the user's back when said body harness is worn, said back panel having a top edge, a bottom edge, a first lateral edge and a second lateral edge, each of said first lateral edge and said second lateral edge curving inwardly between said bottom edge and said top edge such that said bottom edge has a length being greater than a length of said top edge, said body harness comprising:
a chest strap having a first end, a second end, a lower edge and an upper edge, said chest strap being elongated between said first end and said second end wherein said chest strap is configured to be worn around the user's chest, said lower edge being attached to said top edge of said back panel;
a pair of chest couplers, each of said chest couplers being coupled to a respective one of said first end and said second end of said chest strap, each of said chest couplers being releasably matable to each other for retaining said chest strap around the user's chest;
a first coupler being coupled to and extending upwardly from said upper edge of said chest strap, said first coupler being spaced from said first end of said chest strap;
a second coupler being coupled to and extending upwardly from said upper edge of said chest strap, said second coupler being spaced from said second end of said chest strap;
a first shoulder strap being coupled to and extending upwardly from said upper edge of said chest strap wherein said first shoulder strap is configured to be worn over a respective one of the user's shoulders, said first shoulder strap having a distal end with respect to said chest strap;
a first shoulder coupler being coupled to said distal end of said first shoulder strap, said first shoulder strap being oriented to extend cross wise across the user's chest having said first shoulder coupler releasably engaging said second coupler;
a second shoulder strap being coupled to and extending upwardly from said upper edge of said chest strap wherein said second shoulder strap is configured to be worn over a respective one of the user's shoulders, said second shoulder strap having a distal end with respect to said chest strap;
a second shoulder coupler being coupled to said distal end of said second shoulder strap, said second shoulder strap being oriented to extend cross wise across the user's chest having said second shoulder coupler releasably engaging said first coupler;
a waist strap having a primary end, a secondary end, an upper edge and a lower edge, said waist strap being elongated between said primary end and said secondary end wherein said waist strap is configured to be worn around the user's waist, said upper edge being coupled to said bottom edge of said back panel;
a pair of waist couplers, each of said waist couplers being coupled to a respective one of said primary end and said secondary end of said waist strap, each of said waist couplers being releasably matable to each other for retaining said waist strap around the user's waist;
a pair of leg straps, each of said leg straps being coupled to and extending downwardly from said lower edge of said waist strap wherein each of said leg straps is configured to extend downwardly along a back side of a respective one of the user's legs, each of said leg straps having a distal end with respect to said lower edge of said waist strap; and
a pair of leg strap couplers, each of said leg strap couplers being coupled to said distal end of a respective one of said leg straps;
a pair of thigh cuffs each being configured to be worn around a respective one of the user's thighs, each of said thigh cuffs being attachable to said body harness thereby inhibiting said back panel from moving beyond a pre-determined distance from said thigh cuffs wherein said pair of thigh cuffs is configured to assist the user with standing upright, each of said thigh cuffs having a first surface, a second surface and a perimeter edge extending between said first surface and said second surface, said perimeter edge having a first lateral side, a second lateral side, a top side and a bottom side, each of said thigh cuffs being rolled onto themselves to form a cylinder, said top side having a length being greater than a length of said bottom side such that each of said cylinder formed by said thigh cuffs tapers between said top side and said bottom side wherein each of said thigh cuffs is configured to conform to the shape of the user's thighs;
a pair of first mating members, each of said first mating members being coupled to said first surface of a respective one of said thigh cuffs, each of said first mating members being aligned with said first lateral side of said perimeter edge of said respective thigh cuff;
a pair of second mating members, each of said second mating members being coupled to said second surface of a respective one of said thigh cuffs, each of said second mating members being aligned with said second lateral side of said perimeter edge of said respective thigh cuff, said first mating member on a respective one of said thigh cuffs being matable to said second mating member on said respective thigh cuff;
a pair of first heel strap mating members, each of said first heel strap mating members being coupled to said first surface of a respective one of said thigh cuffs, each of said first heel strap mating members extending between said top side and said bottom side of said perimeter edge of said respective thigh cuff;
a pair of thigh couplers, each of said thigh couplers being coupled to said top side of said perimeter edge of a respective one of said thigh cuffs, each of said thigh couplers being releasably matable to a respective one of said leg strap couplers;
a pair of heel straps, each of said heel straps being releasably matable to a respective one of said thigh cuffs, each of said heel straps having a loop being integrated therein wherein said loop on each of said heel straps is configured to be positioned around a respective one of the user's heels thereby enhancing stability of the user for standing upright, each of said heel straps having an upper end, a lower end and a primary surface extending between said upper end and said lower end, each of heel straps having a stirrup being coupled to said lower end having said stirrup on each of said heel straps defining said loop wherein said stirrup is configured to be positioned around the respective user's heel; and
a pair of second heel strap mating members, each of said second heel strap mating members being coupled to said primary surface of a respective one of said heel straps, each of said second heel strap mating members being matable to a respective one of said first heel strap mating members.
